(19) United States
(12) Patent Application Publication
(10) Pub. No.: US 2005/0282896 A1
(43) Pub. Date: Dec. 22, 2005

(54) Methods of cardioprotection using dichloroacetate in combination with an inotrope

(75) Inventors: Gary D. Lopaschuk, Edmonton (CA); Ruth Collins-Nakai, Edmonton (CA)

Correspondence Address: PILLSBURY WINTHROP SHAW PITTMAN LLP, ATTENTION: DOCKETING DEPARTMENT, 11682 EL CAMINO REAL, SUITE 200, SAN DIEGO, CA, 92130, US

(73) Assignee: The University of Alberta, Edmonton (CA)

(21) Appl. No.: 11/013,666

(22) Filed: Dec. 15, 2004

Related U.S. Application Data
(63) Continuation-in-part of application No. 10/778,791, filed on Feb. 13, 2004.
Application No. 10/778,791 is a continuation of application No. 10/268,069, filed on Oct. 7, 2002, now Pat. No. 6,693,133.

(51) Int. Cl. A61K 31/19

(52) U.S. Cl. 514/557

(57) ABSTRACT

The present invention provides compositions and methods for maintaining or improving cardiac function by administering a cardioprotective amount of dichloroacetate (DCA) and an inotropic drug. Also provided are dosage protocols and pharmaceutical compositions for use in these methods.

### CROSS-REFERENCE TO RELATED APPLICATIONS

This application is a continuation-in-part of U.S. Ser. No. 10/778,791, filed Feb. 13, 2004 which is a continuation of U.S. Ser. No. 10/268,069, filed Oct. 7, 2002, now U.S. Pat. No. 6,693,133.

### BACKGROUND AND INTRODUCTION TO THE INVENTION

There is a need for methods of protecting the heart from injury, which may occur due to ischemic incidents and during reperfusion following ischemia, and maintaining cardiac function at a predetermined level thereafter.

Clinically, ischemia-reperfusion may occur in the setting of cardiac surgery. In order to perform many surgical procedures it is necessary to interrupt coronary blood flow resulting in ischemia to the heart. This ischemia not only limits the time available for the surgical procedure, it can also result in contractile dysfunction upon restoration of coronary flow. This is not only a problem in the adult patient undergoing coronary artery bypass surgery (“CABG”) or other surgical procedures, it is also a significant clinical problem during surgical heart procedures to correct congenital heart defects in neonates.

Current therapies aimed at improving contractile function following cardiac surgery in adult, pediatric and neonatal patients often involve the use of inotropes (e.g., calcium, dopamine, epinephrine, ephedrine, phenylephrine, dobutamine) in an attempt to increase contractile function. Although inotropic agents such as dobutamine have been reported to increase myocardial stroke volume and work, they also have been reported to increase myocardial oxygen consumption, and therefore may not enhance mechanical efficiency (1). In fact, the potential for inotropes to increase oxygen consumption to a greater extent than contractile function has been termed an oxygen wasting effect (2, 3). Inotropic drugs are also reportedly associated with increases in intracellular calcium concentration and heart rate, which may also be potentially harmful, especially in hearts with impaired energy balance (4).

### SUMMARY OF THE INVENTION

The present invention is directed to methods of maintaining and improving cardiac function during and following an ischemic event and during reperfusion by administration of dichloroacetate (“DCA”) in combination with an inotropic drug. According to one aspect the methods of the present invention improve cardiac functional recovery and metabolism after an ischemic event, such as surgical heart procedures (including cardiopulmonary bypass and congenital lesions) in patients, as well as cardiovascular disorders such as hemorrhagic shock, hypoxia and trauma.

According to an aspect of the present invention, combination therapy of DCA with an inotropic drug will enable administration of a lower dose of inotropic drug needed to maintain contractile function post-surgery.

One aspect of the present invention is directed to a method of decreasing the amount of inotropic drug needed to maintain a predetermined level of cardiac function in a patient which comprises administering to said patient a cardioprotective amount of dichloroacetate (DCA). According to this aspect, DCA may be administered as a bolus of at least about 50 mg/kg. According to one embodiment, DCA is administered in a bolus of at least about 100 mg/kg. According to one embodiment, administration of the DCA bolus is followed by an infusion of about 12.5 mg/kg/hour DCA for at least about 24 hours. According to another embodiment, the DCA bolus is followed by an infusion of about 25 mg/kg/hour DCA for at least about 24 hours.

According to another aspect of the present invention, provided is a method of maintaining cardiac function at a predetermined level in a patient after cardiac surgery and decreasing said patient's need for inotropes which comprises administering to said patient DCA in a bolus of at least 50 mg/kg followed by infusion of at least about 12.5 mg/kg/hour or alternatively at least about 25 mg/kg/hour for at least about 24 hours. According to one embodiment of this aspect, DCA is administered in a bolus of at least about 100 mg/kg. According to this embodiment, administration of the DCA bolus is followed by infusion of DCA of at least about 12.5 mg/kg/hour for at least about 24 hours. Alternatively, administration of the DCA bolus is followed by infusion of DCA of at least about 25 mg/kg/hour for at least about 24 hours.

In an alternate aspect, the present invention provides an improved method of maintaining cardiac function at a predetermined level in a patient in need of treatment while decreasing inotropic drug requirements, wherein the improvement comprises administering DCA within 15 minutes of administering said inotropic drug.

In another aspect, the present invention is directed to a method of decreasing the inotrope score in a patient who has undergone cardiac surgery which comprises administering a cardioprotective amount of DCA.

Please note that while the present invention is not limited to a particular dose level of DCA, doses and dosing protocols are suitable for use according to the methods of the present invention include the following. According to one aspect, DCA is administered continuously and a plasma level of at least about 1 mM is maintained in the patient for at least about 24 hours. According to one embodiment, a plasma level of at least about 1 mM, alternatively from about 1 mM to about 2 mM is maintained. The plasma level is maintained for at least about 1 hour, alternatively at least about 24 hours. According to an aspect of this embodiment, DCA is administered as a bolus before beginning the continuous administration of DCA. Suitable bolus doses are at least about 50 mg/kg, alternatively at least about 100 mg/kg. Suitable dose ranges for the bolus include at least about 50 mg/kg, alternatively from about 50 mg/kg to about 100 mg/kg or more. Suitable dose ranges for DCA infusion include at least about 12.5 mg/kg/hour, alternatively at least about 25 mg/kg/hour. The DCA infusion may be maintained for a prolonged period of time, suitably for at least about 10 hours, alternatively DCA infusion takes place for about 24 hours or more.

According to one aspect, the present invention provides DCA and inotropic drug to be administered in combination with each other, as in a single solution comprising DCA and inotrope. This combination method of administration allows decreasing the inotrope score in a patient who has undergone cardiac surgery wherein DCA is administered in a cardioprotective amount. In a further aspect of the invention, the method entails the administration of a bolus of DCA as described herein followed by administration of the combination intravenously, such as by intravenous infusion.

According to another aspect of the invention, provided is a pharmaceutical combination comprising a cardioprotective amount of DCA and an inotropic drug, the inotropic drug may be present at a therapeutically effective concentration to provide a lower dose of inotropic drug than the dose of inotropic drug that would be therapeutically effective in the absence of DCA.

According to one aspect, the present invention is directed to a method of maintaining or improving cardiac function during or following a cardiac function disturbing event or a cardiac metabolism disturbing event in a patient which comprises administering to said patient a cardioprotective amount of DCA and an inotropic drug.

In an alternate aspect, the present invention is directed to methods of maintaining cardiac function at a predetermined level in a patient during or following a cardiac function or cardiac metabolism disturbing event and decreasing the patient's need for inotropic drugs which comprises administering to said patient a cardioprotective amount of DCA.

In another aspect, the present invention is directed to methods of treating an ischemic, hypoxic or metabolic event or an event which results in cardiac dysfunction in a patient which comprises administering said patient a cardioprotective amount of DCA and an inotropic drug.

According to a further aspect of the methods of the present invention, the inotropic drug is administered with arginine or an agent which increases arginine levels or stimulates arginine release.

Additionally, the present invention provides pharmaceutical compositions suitable for use in to the methods of the present invention. Thus, provided are pharmaceutical compositions comprising a cardioprotective amount of DCA and an inotropic drug. Suitable inotropic drugs include, but are not limited to, agents selected from the group consisting of a beta-adrenergic receptor agonist, a PDE3 inhibitor, an agent which increases cAMP levels; a Na⁺, H⁺ exchange inhibitor; a Na⁺, Ca²⁺ exchange blocker; a non-adrenergic vasopressor, an alpha-2-adrenergic agonist, an ET-1 antagonist; an ion channel blocker; an ACE inhibitor; a Na⁺ K⁺-ATPase inhibitor, a Na⁺, K⁺ exchange inhibitor; a cardiac glycoside; a sympathomimetric and other agents having a positive inotropic effect which are known to those of skill in the art.

According to one embodiment, the composition may further comprise a beta-adrenergic receptor agonist. According to an alternate embodiment, the composition may further comprise an agent which increases arginine levels.

Also included within the present invention are kits which comprise a pharmaceutical composition as described herein. The kit may also comprise a label or packaging insert containing instructions for use.

Definitions:

“Inotrope” or “inotropic drug” refers to a member of a class of pharmaceutical agents that have a positive inotropic effect, including agents which increase the contractility of cardiac muscle, have a strengthening effect on the heart, or increase cardiac output. These agents include cardiac glycosides, sympathomimetics, beta-adrenergic receptor agonists, phosphodiesterase 3 (PDE3) inhibitors, calcium-sensitizers; sodium, calcium (Na⁺/Ca²⁺) exchange blockers; sodium potassium (Na⁺/K⁺) exchange inhibitors; Na⁽⁺⁾, K⁽⁺⁾-ATPase inhibitors; sodium hydrogen (Na⁺, H⁺) exchange inhibitors; alpha-2-adrenergic agonists; non-adrenergic vasopressors; endothelin 1 (ET-1) antagonists; angiotensin converting enzyme (ACE) inhibitors; agents which increase cyclic adenosine monophosphate (cAMP) levels; agents which increase L-arginine levels or release of L-arginine, and other agents having a positive inotropic effect as noted in the “Detailed Description of the Invention” or known to those of skill in the art. Inotropes or inotropic drugs conventionally used to maintain cardiac function and contractility include dobutamine, epinephrine, dopamine, norepinephrine, phenylephrine, phentolamine, digoxin, amrinone, and other agents known to those in the art and include, without limitation, the inotrope or inotropic drugs mentioned in the “Detailed Description of the Invention” as well as others known to those of skill in the art. Indications where inotropes or inotropic drugs may be used to treat patients include after myocardial infarct, during and after cardiac surgical procedures, in shock or in congestive heart failure.

The term “positive inotropic effect” refers to an agent having a positive effect on the force of muscular contractions of cardiac tissue and includes agents that increase the contractility of cardiac muscle, that have a strengthening effect on the heart or that can increase cardiac output.

The term “cardiac event” refers to an event in a patient where cardiac function changes from what had been the patient's baseline function. Cardiac events include events which disturb cardiac function and events which disturb cardiac metabolism. Examples of cardiac events include, but are not limited to, ischemic events, hypoxic events, acute myocardial infarction, acute heart failure, congestive heart failure, cardiomyopathy, diabetic cardiomyopathy, acute coronary syndrome, angina, post-percutaneous transluminal coronary angioplasty, shock, hemorrhagic shock, trauma, sepsis, cardiac surgical procedures (including CAGB), HIV, malaria, cancer chemotherapy, hypertension, pulmonary hypertension, and other conditions known to those of skill in the art.

### BRIEF DESCRIPTION OF THE DRAWINGS

FIG. 1 depicts a chart noting the pre-op and post-op cardiac medications used for the patients in the study described in Example A.

FIG. 2 depicts a graph of pyruvate dehydrogenase activity (PDH) after administration of a 50 mg/kg bolus of DCA or placebo. See Example A.

FIG. 3 depicts a graph of plasma levels of acetate following infusion of placebo or 50 mg/kg DCA via cardiac bypass pump in the study of Example A.

FIG. 4 depicts a graph for the inotrope score for patients treated with DCA (50 mg/kg bolus) versus placebo and the relative decrease in 1 hour inotrope score of DCA treated patients compared to placebo. See Example B.

FIG. 5 depicts a graph of decrease in ICU time for patients treated with DCA (50 mg/kg bolus) as compared to placebo. See Example B.

FIG. 6 depicts a graph of the decrease in ventilator time for patients treated with DCA (50 mg/kg bolus) versus placebo. See Example B.

FIG. 7A depicts a summary of patients in the study of Example C treated pre-op or post-op with inotropes.

FIG. 7B depicts a list of hemodynamic drugs routinely administered pre-op or post-op to cardiac surgery patients such as the patients of the studies described in Examples B and C.

FIG. 8 depicts a graph of the effects on inotrope score of administration of a 50 mg/kg bolus of DCA followed by a 25 mg/kg/hour infusion versus placebo in post-heart surgery patients. See Example C.

FIG. 9 depicts a graph of the effects on inotrope score of DCA administration as a 100 mg/kg bolus and 12.5 mg/kg/hour infusion versus placebo post-surgery in pediatric patients. See Example C.

FIG. 10 depicts a graph of effects on reducing ICU time of DCA administration as a 50 mg/kg bolus and 25 mg/kg/hour infusion post-surgery in patients as compared to placebo. See Example C.

FIG. 11 depicts a graph of the effects on reducing ICU time for patients with DCA treatment as 100 mg/kg bolus and 12.5 mg/kg/hour infusion post-surgery as compared with placebo. See Example C.

FIG. 12 depicts a graph of the effects on reducing ventilator time for patients with DCA administration as a 50 mg/kg bolus and 25 mg/kg/hour infusion post-surgery as compared with placebo. See Example C.

FIG. 13 depicts a graph of the effects on ventilator time for patients with DCA treatment as a 100 mg/kg bolus and 12.5 mg/kg/hour infusion post-surgery as compared to placebo. See Example C.

### DETAILED DESCRIPTION OF THE INVENTION

As noted, in one aspect, the present invention provides methods of maintaining or improving cardiac function following a cardiac function disturbing event or a cardiac metabolism disturbing event by administering a cardioprotective amount of DCA and an inotropic drug. Such cardiac function distrubing events and/or cardiac metabolism disturbing events include an ischemic event (such as acute myocardial infarction), acute heart failure, an event caused by hemorrhagic shock, hypoxia or trauma; cardiomyopathy (including diabetic cardiomyopathy); an event due to an HIV infection; an event due to malaria; acute coronary syndrome (including events which are post-AMI, post PTCA or angina); shock (including events where shock is secondary to hemorrhage, hypoxia, trauma or sepsis); and events associated with diabetes; events following or resulting from cancer chemotherapy and other events resulting from disturbances in cardiac function or cardiac metabolism.

The present invention provides methods of maintaining cardiac function at a predetermined level during or following a cardiac function disturbing event or a cardiac metabolism disturbing event and of decreasing the patient's need for inotropic drugs by administering a cardioprotective amount of DCA. Suitably, DCA and an inotropic drug are administered in combination. According one embodiment, DCA is administered within about 15 minutes of administering the inotropic drug. Suitable inotropic drugs include dosutamine, epinephrine, dopamine, morepinephine, phentolamine, digoxin, amrinone, milrone, enoximore, as well as other inotropic drugs described herein or known to those of skill in the art.

Suitable dosing protocols for these methods include administering DCA in a bolus of at least about 50 mg/kg, or advantageously about 100 mg/kg or more. The DCA bolus is followed by continuous infusion of DCA of at least about 12.5 mg/kg/hour, or advantageously at least about 25 mg/kg/hour, for at least an hour. Suitably DCA is infused for at least about 10 hours or alternatively for at least about 24 hours or more.

Accordingly to one aspect of these methods, the inotropic drug is selected from the group consisting of a beta-adrenergic receptor agonist, a phosphodiesterase 3 (PDE3) inhibitor, an agent which increases cyclic AMP levels; a sodium, hydrogen (Na⁺, H⁺) exchange inhibitor; and a sodium, calcium (Na⁺, Ca²⁺) exchange blocker. Where the inotropic drug is a PDE3 inhibitor, the methods may further comprise administering a beta-adrenergic receptor agonist. According to alternate aspects, the inotropic drug may be either a non-adrenergic agonist, an endothelin 1 (ET-1) antagonist, an ion channel blocker, a calcium-sensitizer, a calcium channel blocker, an angiotensin converting enzyme (ACE) inhibitor, a PDE3 inhibitor (optimally administered with a beta-adrenergic receptor agonist), an agent which increases cyclic AMP levels; an Na⁺, K⁺-ATPase inhibitor, a cardiac glycoside or other agent having a positive inotropic effect.

The present invention provides methods of treating an ischemic, hypoxic or metabolic event or an event resulting in cardiac dysfunction by administering a cardioprotective amount of DCA and an inotropic drug. Such an event may be due to a surgical procedure, percaneous intervention, acute myocardial infarction or an acute coronary syndrome (ACS). Acute coronary syndromes include cardiogenic shock, hemorrhagic shock and trauma. Alternatively, such an event may result from, sepsis, HIV or malaria. The event to be treated may follow cancer chemotherapy. Such event may be due to or result from, angina, hypertension, pulmonary hypertension, diabetic cardiomyopathy, cardiomyopathy, congestive heart failure or diabetes. The event to be treated may result in cognitive impairment. According to one embodiment, the dosing protocol for DCA comprises administering a bolus of DCA, followed by continuous infusion of DCA for a period of time. DCA may be administered in a bolus of at least about 50 mg/kg, and suitably in a bolus of about 100 mg/kg or more. DCA may be infused at a rate at least about 12.5 mg/kg/hour, and suitably at least about 25 mg/kg/hour. DCA may be infused for an extended period of time, for example for at least about 1 hour, alternatively about 10 hours or more or about 24 hours or more.

Pharmaceutical compositions suitable for use accordingly to the present invention include a cardioprotective amount of DCA and an inotropic drug. The composition suitably comprises an amount of inotropic drug effective to maintain or improve cardiac function when in combination with the cardioprotective amount of DCA. Suitable inotropic drugs for use in the pharmaceutical compositions include those described herein as well as other inotropic drugs or agents having a positive inotropic effect when administered to a patient which are known in the art.

Cardiac Metabolism

Under normal aerobic conditions, oxidation of fatty acids is the predominant source of energy (ATP) production in the heart, with a lesser contribution being derived from lactate and glucose. However, during ischemia (such as occurs during cardiac surgery), when the supply of oxygen becomes limiting, anaerobic glycolysis assumes a more important role, and fatty acid and carbohydrate oxidation decrease (5, 6). During reperfusion following ischemia, ATP production, tricarboxylic acid (TCA) cycle activity and oxygen consumption rapidly recover. Fatty acid oxidation also quickly recovers providing over 90% of the overall ATP production (7, 8). The reason for this increase in fatty acid oxidation is reportedly due both to ischemic-induced alterations in the control of myocardial fatty acid oxidation (9, 10), as well as an increase in circulating fatty acid levels (11, 12). The use of inotropes with adrenergic agonist properties can also contribute to these high plasma levels of fatty acids. This excessive use of fatty acids by the heart following ischemia can have adverse effects on both cardiac function and cardiac efficiency.

The availability of different energy substrates and the type of energy substrate used by the heart can have profound effects on cardiac functional recovery during and following an ischemic episode. Specifically, high rates of fatty acid oxidation may contribute to a marked decrease in cardiac efficiency secondary to inhibition of glucose oxidation (5, 6, 7). However, if glucose oxidation is stimulated during reperfusion, a significant increase in cardiac efficiency results, with a parallel improvement in cardiac function (11). This is partly due to a decreased requirement of oxygen to produce equivalent amounts of ATP (7, 13). Stimulating glucose oxidation also decreases the production of protons in the heart, therefore decreasing the amount of ATP necessary to maintain ionic homeostasis in the heart.

In fetal life, glycolysis and lactate oxidation are the major sources of ATP production. However, following birth there is a rapid maturation of fatty acid oxidation, which rapidly becomes the predominant source of ATP production in the newborn heart (13, 19, 20). Under aerobic conditions, glucose oxidation rates are lower in neonatal hearts compared with adult hearts (21, 22). Simultaneous measurement of both glycolysis and glucose oxidation in neonatal hearts has demonstrated that glycolytic rates are much greater than rates of glucose oxidation, suggesting low flux through pyruvate dehydrogenase (PDH), the rate-limiting enzyme for glucose oxidation (21). Therefore, when the newborn heart is subjected to ischemia-reperfusion injury during open heart surgery, the increase in fatty acid oxidation may be particularly detrimental, since the glucose oxidation pathway in these hearts has not completely matured. Studies in immature rabbit hearts have shown that addition of pyruvate, a substance that stimulates PDH activity, significantly increases aortic flow, cardiac work, and developed pressure (23). Based on these studies, we believe that a metabolic therapy, which stimulates glucose oxidation at the expense of fatty acid oxidation, would enhance cardiac recovery following ischemia.

Cardioprotective Effects of Dichloroacetate on the Heart

We have found dichloroacetate (DCA) to be particularly effective at stimulating glucose oxidation in the heart. DCA has been reported to stimulate pyruvate dehydrogenase (PDH), the rate-limiting enzyme for glucose oxidation in the heart (14, 15). This stimulation appears to occur via DCA inhibition of PDH kinase, which normally phosphorylates and inhibits PDH. In experimental studies on isolated rat hearts, we showed that DCA dramatically improves functional recovery and cardiac efficiency during reperfusion of hearts following a severe episode of ischemia (9, 16, 17). This beneficial effect of DCA is due to a dramatic stimulation of glucose oxidation and a switch in energy substrate use by the heart from fatty acid oxidation towards glucose metabolism (15, 7). DCA also dramatically decreases proton production in the reperfused ischemic heart, which is a major reason for the DCA-induced improvement in cardiac efficiency during reperfusion (16).

Since DCA has demonstrated such dramatic effects in our studies on cardioprotective effects on the ischemic heart, it may be of clinical use in maintaining and improving cardiac function (including contractility) in the setting of cardiac surgery both for the adult and pediatric patient. Plasma levels of fatty acids have been observed to increase significantly during reperfusion following cardiac surgery. This increase is observed to be highest in pediatric patients, including patients as young as three weeks of age (10). Elevations in free fatty acids may result in an increase in myocardial oxygen consumption, which may potentiate ischemic injury (11).

Inotropes are frequently administered to patients to improve contractile function of the heart following cardiac surgery. However, some effects of inotropes may not be desirable. For example, epinephrine, an inotropic agent, has been reported to increase the uncoupling between glycolysis and glucose oxidation resulting in a significant increase in proton production from glucose metabolism (24). This potentially may accelerate acidosis during the reperfusion period, at a time when the heart is trying to clear a preexisting proton load produced during ischemia, and would be another undesirable effect of inotrope use (8, 1).

While not wanting to be bound to a particular theory, we believe that by stimulating glucose oxidation, administration of DCA lessens the need for inotropes (or dose of inotrope) and other hemodynamic drugs used post-operatively. We have shown that DCA is cardioprotective in adults, pediatric patients, and neonates undergoing open heart cardiac surgical procedures. The present examples describe studies that determine that DCA when used in combination with inotropes lessens the dose of inotrope needed.

In one aspect, the present invention is directed to the use of dichloroacetate (DCA) to improve cardiac functional recovery and metabolism after open heart surgical procedures (cardiopulmonary bypass and congenital lesions) in patients and to decrease the need for administering of inotropes and if inotropes are administered, decrease the dose of inotrope needed to maintain cardiac function (including contractility) at a desired predetermined level. Administration of DCA lessens the need for inotropes and other hemodynamic agents. As a result, combination therapy with DCA will allow for a lowering of the amount and doses of inotropes used.

We believe that pediatric patients receive even greater benefits from DCA during cardiac surgery because, as previously noted, they have the highest fatty acid levels during and after cardiac surgery accompanied by the lowest rates of glucose oxidation. In a study of 40 pediatric patients (age 0.03-15.1 years) requiring open heart surgery (see Example B), DCA was given as a bolus dose of 50 mg/kg into the aortic root just prior to the release of the cross clamp. One-hour Inotrope Score was significantly lower in the DCA group compared to placebo (which indicated better cardiac function). ICU days and ventilator hours were also lower in the DCA group. This study demonstrated that DCA, when used in combination with inotropes, will lessen the requirements for inotropes in the immediate post-surgery period.

Use of DCA as a Cardioprotective Agent and to Decrease the Need for Inotropes or Inotropic Drugs

The studies described in Example A demonstrate that DCA administration increases PDH activity in the human heart and improves carbohydrate oxidation.

In Example A, studies in 18 adult Coronary Artery Bypass Graft (CABG) patients demonstrated that giving DCA as a bolus was effective in producing the desired metabolic effects of DCA. Cardiac PDH enzyme activity following surgery was increased significantly following administration of DCA. As well, DCA also significantly decreased plasma lactate levels.

In the studies described in Examples B and C we observed that DCA administered as a bolus dose post-surgery to pediatric patients undergoing cardiac surgery significantly lowered the dose of inotropes required to sustain contractile function and decreased the time spent in the Intensive Care Unit (ICU).

When DCA was administered using a bolus and infusion protocol to maintain therapeutic levels of DCA over a 24 hour period during reperfusion for surgical heart procedures, the therapeutic benefits of DCA were sustained in the presence of other clinically recommended hemodynamic drugs, the requirements for inotropes were decreased, and the patients' time spent on the ventilator and in the ICU was significantly decreased.

In Example B, where DCA was administered as a bolus dosing protocol, the clinical benefit of DCA was demonstrated in a study which consisted of a 40 pediatric patients study for surgical heart procedures. Data from this trial revealed that patients treated with DCA had a significantly reduced Inotrope Score, had reduced time in ICU and had reduced time on the ventilator as compared to patients treated with placebo. The results observed after administration of DCA as a bolus of 50 mg/kg in the study described in Example B encouraged us to proceed with the DCA protocol used for the study described in Example C.

A dose range for DCA of about 1 mM has been shown to be effective in increasing PDH levels and improving myocardial function in isolated perfused hearts. (This dose range was also supported by data from the study described in Example A using a bolus administration of 50 m/kg DCA.) The bolus and infusion administration in the study described in Example C provided the therapeutic benefits of DCA at a DCA therapeutic level in blood plasma of 1 mM (7, 9, 16, 17) during the critical 24 hour period post-surgery. Using a bolus and infusion protocol, data from the study described in Example C (which consisted of 51 pediatric patients) revealed that such treatment resulted in a reduced need for inotropic drugs. (As noted in Example C, the final results were based on 47 patients, 51 patients less 4 infusion pump failure cases).

In the study described in Example C, the DCA protocols used two different dosing administrations in the presence of clinical recommended therapeutic levels of hemodynamic drugs: Group A was originally given a bolus of 50 mg/kg and an infusion of 25 mg/kg/hr; and Group B was given a bolus of 100 mg/kg and an infusion of 12.5 mg/kg/hr. (The cardiac surgeon in the study described in Example C had good results and therefore used less inotropes to maintain cardiac index in all patients.) In the study described in Example C, the DCA therapeutic range level of the DCA patients in both Groups A and B, showed benefits at DCA therapeutic plasma levels 0.229 mM to 2.22 mM at the 1 to 6 hour interval, and from 1.74 mM to as high as 3.9 mM at the 24 hour interval (Table I). There were 11 DCA patients in each of Groups A and B, and noted below in Table I as n=the number of DCA patients where the DCA blood plasma levels measured at each interval.

TABLE I
Average DCA Plasma Levels (Example C)

| | mM at | mM at | mM at | mM at |
|---|---|---|---|---|
| | 1 hr | 6 hr | 12 hr | 24 hr |
| Group A 50 mg/kg | .460 | 1.134 | 1.771 | 2.724 |
| bolus and 25 | (n = 11) | (n = 10) | (n = 9) | (n = 11) |
| mg/kg/hr infusion | | | | |
| Group B 100 mg/kg | 1.131 | .894 | 1.427 | 2.231 |
| bolus and 12.5 | (n = 11) | (n = 11) | (n = 11) | (n = 10) |
| mg/kg/hr infusion | | | | |

The DCA therapeutic optimum means for the different intervals from the study described in Example C were based on the DCA patient outcomes with the greatest degree of clinical benefits (cardiac index, ICU and ventilator time) as compared to placebo. These DCA plasma range outcomes were from both the simple open heart surgery and complex open heart surgery patients—at the 1 hour interval from Group B, and at the 12 and the 24 hour intervals from Group A. The optimum DCA therapeutic dose level average means are as summarized below in Table II.

TABLE II
Average Optimum Mean of DCA Plasma Levels (Example C)
Group A and Group B n = data from DCA patients
numbers with Greatest Degree of Clinical Benefits

| | mM at | mM at | mM at | mM at |
|---|---|---|---|---|
| | 1 hr | 6 hr | 12 hr | 24 hr |
| Group A 50 mg/kg | 1.0 | 0.916 | 1.523 | 2.288 |
| bolus and 25 | | (m = 5) | (m = 5) | (m = 6) |
| mg/kg/hr infusion | | | | |
| Group B 100 mg/kg | 1.012 | 1.0 | 1.0 | 1.0 |
| bolus and 12.5 | (n = 7) | | | |
| mg/kg/hr infusion | | | | |

The known DCA therapeutic dose level mean of 1 mM was observed in Group B at the 1 to 6 hour interval (with a DCA plasma level range of 0.229 mM to 2.22 mM) A different optimum DCA therapeutic dose level at 2.29 mM means was observed from Group A at the 24 hour period (with a DCA plasma level range of 1.73 mM to 3.91 mM).

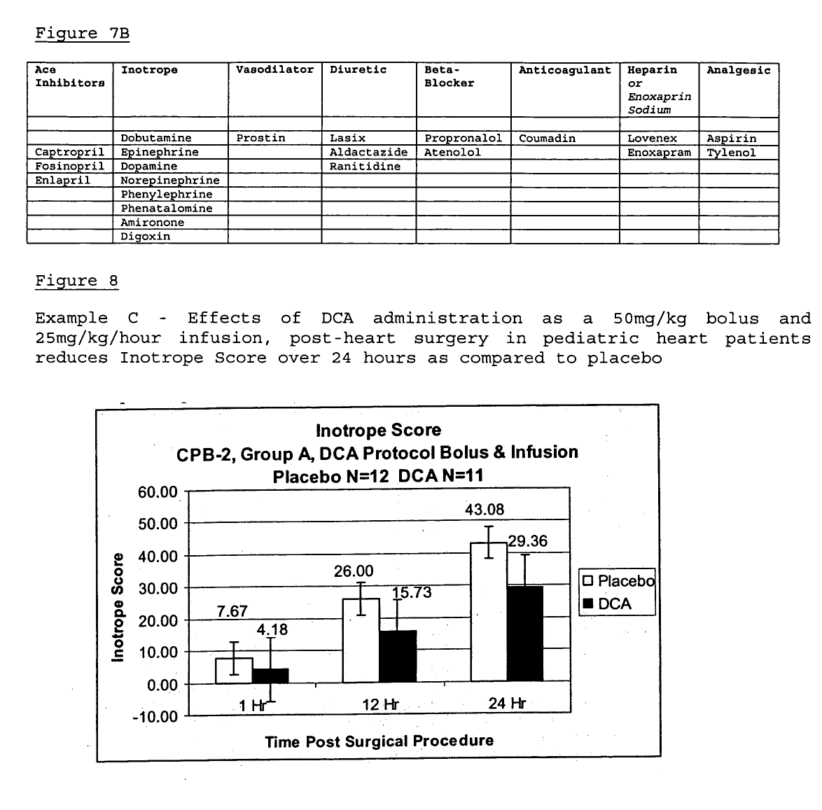

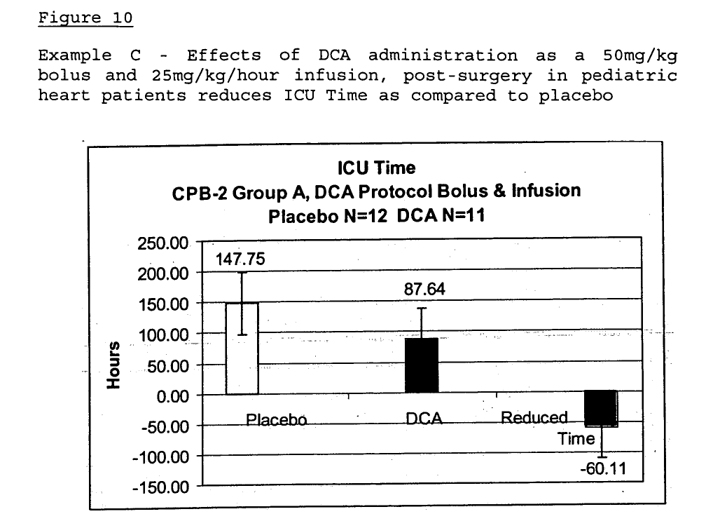

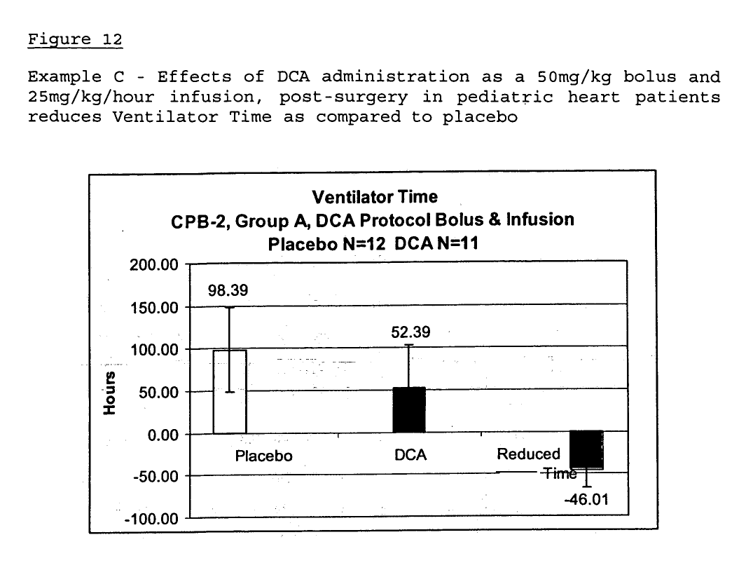

The resulting data in the Group A protocol of a bolus of 50 mg/kg and an infusion of 25 mg/kg/hour post-surgical heart procedure for 23 patients reduced the time in ICU (FIG. 10) post-surgical procedure by 60 hours (a 41% decrease) as compared to placebo. The reduction of Inotrope Scores (FIG. 8) was by 1 hour at a 50% decrease, and by 12 hours at a 45% decrease and by 24 hours at a 38% decrease as compared to placebo. Ventilator time (FIG. 12) was reduced by 46 hours (a 47% decrease) as compared to placebo.

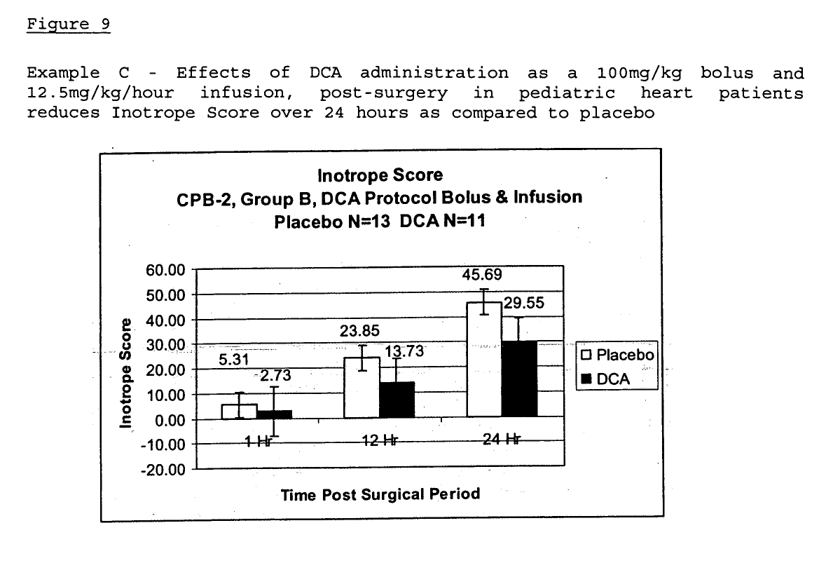

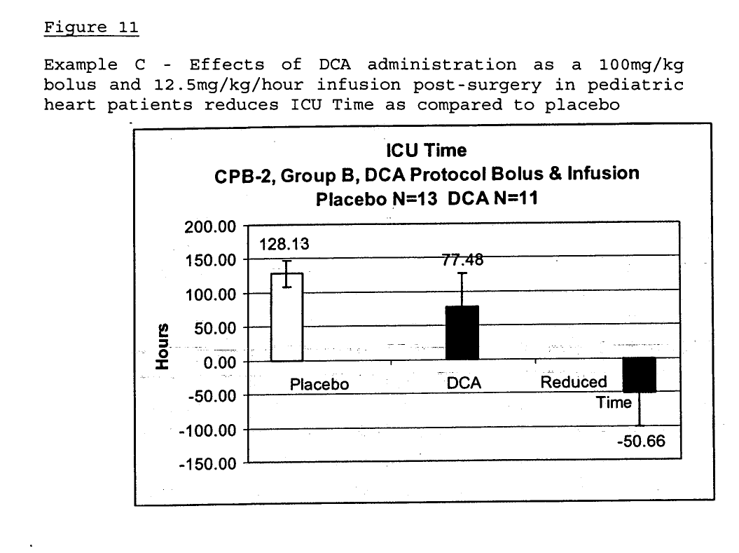

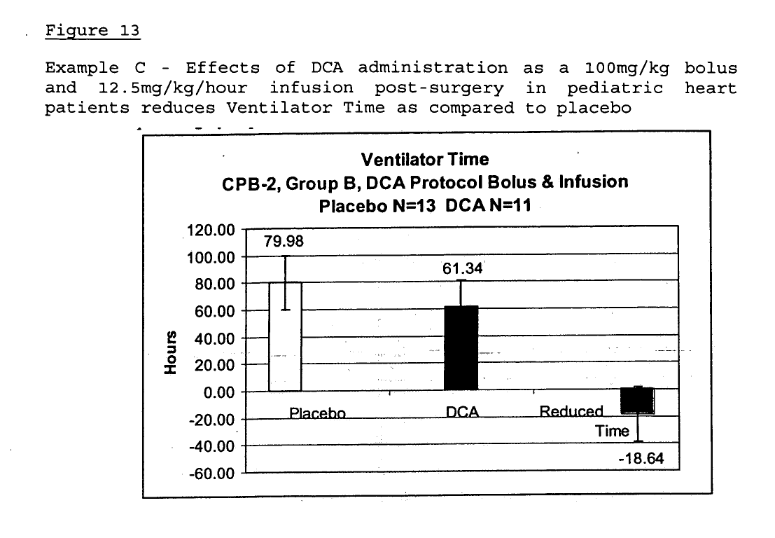

The resulting data in the Group B protocol of a bolus of 100 mg/kg and an infusion of 12.5 mg/kg/hour, post-surgical heart procedure for 24 patients, reduced the time in ICU (FIG. 11), post surgical procedure over the 24 hour period by 50 hours (a 40% decrease) as compared to placebo. The reduction of Inotrope Scores (FIG. 9) averaged by 1 hour at a 57% decrease, and by 12 hours at a 49% decrease, and by 24 hours at a 45% decrease as compared to placebo. Ventilator Time (FIG. 13) was reduced by 19 hours (a 23% decrease) as compared to placebo.

The merit of reducing inotropic drugs with the bolus and infusion administration of DCA is supported by the data from the 1 hour period through the 24 hour period post surgical heart procedures from the study described in Example C.

The data measurement outcomes from in vitro modeling testing (17) indicate that the administration of DCA at a constant therapeutic level of 1 mM maintains its benefits in the presence of clinically high levels of hemodynamic drugs. Administering a constant optimum therapeutic mean level (based on the optimum outcome) of DCA observed in the presence of clinically acceptable lower levels of hemodynamic drugs will provide significant cardioprotective benefits and decrease deleterious effects which may occur with use of such hemodynamic agents. In the studies described in the Examples, we found optimum mean levels for DCA plasma ranges at specific intervals were as to include: 1 mM (0.229 mM to 2.22 mM DCA plasma range) during the 1 to 6 hour period, 1.52 mM (0.38 mM to 3.07 mM plasma range) at the 12 hour interval, and 2.29 mM (1.73 mM to 3.91 mM DCA plasma range) at the 24 hour interval.

Taken together, improving cardioprotective benefits, and improved cardiac function were maintained by using DCA at a constant therapeutic level of about 1 mM in the presence of clinically recommended dose levels of hemodynamic drugs over a 24 hour period. In the presence of clinically high levels of hemodynamic drugs, by using our DCA protocol to maintain a constant therapeutic range of 1 mM at the 1 to 6 hour period, 1.5 mM at the 12 hour interval, and 2.29 mM at the 24 hour interval, improving cardioprotective benefits, and improved cardiac function are also maintained.

Events Treated with DCA and Inotropic Drug Therapy

Cardiac events to be treated with the methods of the present invention include cardiac function disturbing and cardiac metabolism disturbing events which may have a number of causes. These events include ischemic, hypoxic and/or metabolic events or events which result in dysfunction in acute disease indications including cardiac surgical procedures such as CABG, CPB and valvular surgeries, percutaneous interventions (“PCI”), acute myocardial infarction (“AMI”) and Acute Coronary Syndromes (“ACS”) such as cardiogenic shock, hemorrhagic shock and trauma. Certain of these events may be due to pathologic conditions which result in cardiac dysfunction.

Other such events include ischemic, hypoxic or metabolic events or events resulting in cardiac dysfunction in a patient having sepsis, HIV or malaria.

Additional such events include ischemic, hypoxic or metabolic events or events resulting from cardiac dysfunction occurring following cancer chemotherapy. Other events suitable for treatment include ischemic, hypoxic or metabolic events or cardiac dysfunction resulting in cognitive impairment.

Additional events for treatment according to the compositions and methods of the present invention include ischemic, hypoxic or metabolic events or events resulting in cardiac dysfunction in acute or chronic disease indications which include, but are not limited to, unstable or stable angina, hypertension, pulmonary hypertension, diabetic cardiomyopathy, cardiomyopathy, congestive heart failure or diabetes.

Administration and Dosing of DCA

While it is not intended that the present invention be limited by the particular delivery means, one delivery means is an intravenous means, such as that achieved by introduction through an intravenous drip. Other means includes (but is not limited to) delivery with a catheter. Another means involves direct injection into the aorta, for example, with a catheter. Still other routes of administration include subcutaneous, sublingual and oral routes to achieve a decrease in the amount of inotrope needed to maintain a predetermined level of cardiac function.

The particular dosage of DCA is also not intended to be limiting. A variety of temporal protocols is contemplated. Delivery in a bolus as well as continuous delivery is contemplated. In one embodiment, DCA (such as sodium dichloroacetate) is given in a bolus of at least 100 mg/kg of an approximately 100 mg/ml solution (1.0 cc/kg bolus) and, immediately thereafter, dichloroacetate is given as an infusion at approximately 12.5 mg/kg/hr for greater than about 10 hours and, more preferably, is given as an infusion for about 24 hours or more.

According to an alternate embodiment, DCA is given in a bolus of at least about 100 mg/kg and, immediately thereafter DCA is given as an infusion at about 25 mg/kg/hour for greater than about 10 hours and, suitably, about 24 hours or more.

According to one aspect of the present invention, DCA is administered to a patient under conditions such that said subject has a blood (e.g., serum or plasma) concentration of DCA of greater than approximately 200 μM, alternatively greater than 500 μM, and even greater than 1 mM, for a period of time longer than 1 hour, alternatively longer than 6 hours, and even 24 hours or longer. In one embodiment, DCA is delivered as a bolus, followed by continuous administration.

Higher dosages than those noted above may be used. We have not observed DCA to have significant side-effects, although it has been reported that some patients on chronic dosing experience mild drowsiness.

Inotropic Drugs

A number of pharmaceutical agents have been reported as inotropic drugs and have been reported as having exhibited positive inotropic activity when administered to a patient. These inotropic drugs have been reported to have a positive inotropic effect. Such positive inotropic effects have been reported as resulting from one or more of a number of different mechanisms of action. Classes of pharmaceutical agents reported to exhibit a positive inotropic effect include sodium calcium (Na⁺/Ca²⁺) exchange blockers, phosphodiesterase 3 (“PDE3”) inhibiting drugs, calcium-sensitizers, agents which increase cyclic AMP(cAMP) levels, agents which increase intracellular Na⁺, ion channel blockers (including Na⁺ or H⁺ exchange inhibitors and Na⁺ pump inhibitors), sodium potassium (Na⁺, K⁺) exchange inhibitors, alpha-2-adrenergic agonists, endothelin 1 (ET-1) antagonists (or endothelin 1 (ET-1) receptor agonists), calcium channel blockers, angiotensin converting enzyme (“ACE”) inhibitors; Na⁽⁺⁾, K⁽⁺⁾-ATPase inhibitors; cardiac glycosides; sympathomimetics; beta-adrenergic receptor agonists; non-adrenergic vasopressors; and other vasoconstrictor agents.

Examples of ACE inhibitors include quinoprilat.

Examples of alpha-2-adrenergic agonists include clonidine and moxonidine.

Examples of beta-adrenergic receptor agonist inotropic agents include isoproterenol, dopexamine and dobutamine.

Examples of calcium channel blocking agents include diltiazem, nifedipine and other such agents.

Examples of calcium-sensitizers include levosimendan.

Examples of Endothelin 1 (ET-1) antagonists include bosentan and tezosentan.

Examples of Na⁺, H+ exchange inhibitors include cariporide.

Examples of Na⁺, K⁺ ATPase inhibitors include vanadate and 2-methoxy-3,8,9-trihydroxy coumestan.

Examples of non-adrenergic vasopressors include vasopressin.

Examples of sodium pump inhibitors include oubain.

Examples of PDE3 inhibitors include amrinone, milrinone and enoximone.

The listing of classes of pharmaceutical agents having positive inotropic activity is intended as exemplary in nature and other classes of such agents known to those of skill in the art are intended to be included as inotropic drugs. Similarly, with respect to particular agents given as examples of the classes noted above, they are intended as examples only and are not intended as to be an exhaustive listing of suitable agents of a particular class.

Pharmaceutical Compositions and Kits

The invention further provides pharmaceutical compositions comprising a cardioprotective amount of DCA and an inotropic drug or their pharmaceutically acceptable salts, esters or prodrugs. Also contemplated to be within the scope of the present invention are pharmaceutical compositions further comprising a beta-adrenergic receptor agonist. According to an alternate aspect, the pharmaceutical compositions of the present invention further comprise an agent which increases arginine levels.

Pharmaceutical compositions or formulations include compositions and formulations conventionally used in the pharmaceutical arts and may comprise carriers and excipients compatible with oral, intravenous, intramuscular, intraarterial, intracranial, and/or intracavity administration. Suitable pharmaceutical compositions and/or formulations may further compose colloidal dispersion systems, or lipid formulations (e.g., cationic or anionic lipids), micelles, microbeads, etc.

As noted, pharmaceutical compositions of the present invention may comprise pharmaceutically acceptable and physiologically acceptable carriers, diluents or excipients. Examples of suitable carriers, diluents and excipients include solvents (aqueous or non-aqueous), solutions, emulsions, dispersion media, coatings, isotonic and absorption promoting or delaying agents, compatible with pharmaceutical administration, and other commonly used carriers known in the art.

Pharmaceutical compositions may also include carriers to protect the composition against rapid degradation or elimination from the body, and, thus may comprise a controlled release formulation, including implants and microencapsulated delivery systems. For example, a time delay material such as glyceryl monostearate or glyceryl stearate alone, or in combination with a wax, may be employed.

Pharmaceutical compositions can be formulated to be compatible with a particular route of administration. For oral administration, a composition can be incorporated with excipients and used in the form of tablets, pills or capsules, e.g., gelatin capsules. Pharmaceutically compatible binding agents, and/or adjuvant materials can be included in oral formulations. The tablets, pills, capsules, etc., can contain any of the following ingredients, or similar compounds: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; or a flavoring or sweetening agent.

Pharmaceutical compositions for parenteral, intradermal, or subcutaneous administration can include a sterile diluent, such as water, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Pharmaceutical compositions for injection include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). Antibacterial and antifungal agents include, for example, parabens, chlorobutanol, phenol, ascorbic acid and thimerosal. Isotonic agents, for example, sugars, polyalcohols such as manitol, sorbitol, sodium chloride may be included in the composition. Including an agent which delays absorption, for example, aluminum monostearate and gelatin can prolong absorption of injectable compositions.

The pharmaceutical formulations can be packaged in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the pharmaceutical carrier or excipient.

The compositions can be administered by any route compatible with a desired outcome. Thus, routes of administration include oral (e.g., ingestion or inhalation), sublingual, intraperitoneal, intradermal, subcutaneous, intravenous, intraarterial, intracavity, intracranial, and parenteral. The compositions can also be administered using implants and microencapsulated delivery systems.

Compositions, including pharmaceutical formulations can further include particles or a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. Cyclopropanecarboxylic acid, cyclopropanecarboxylic acid and derivatives and modified forms thereof can be entrapped in microcapsules, for example, by the use of hydroxymethylcellulose or gelatin-microcapsules, or poly (methylmethacrolate) microcapsules, respectively, or in a colloid drug delivery system.

The invention provides kits containing a cardioprotective amount of DCA and an inotropic drug, including pharmaceutical formulations, packaged into a suitable set. A kit typically includes a label or packaging insert including instructions for use, in vitro, in vivo, or ex vivo, of the components therein.

The term “packaging material” refers to a physical structure housing the components of the kit, such as DCA and inotropic drug and, if present, pharmaceutically acceptable carrier. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method of the invention.

Kits of the invention therefore can additionally include instructions for using the kit components in a method of the invention. Instructions can include instructions for practicing any of the methods of the invention described herein. Thus, for example, a kit can include DCA and inotropic drug in a pharmaceutical formulation in a container, pack, or dispenser together with instructions for administration to a human subject. Instructions may additionally include indications of a satisfactory clinical endpoint or any adverse symptoms that may occur, or any additional information required by the Food and Drug Administration for use in humans.

A kit may include instructions for administering DCA and inotropic drug in the treatment of an ischemic, hypoxic or metabolic event or an event resulting in cardiac dysfunction in vitro, ex vivo or in vivo. In other embodiments, a kit includes instructions for treating a disorder associated with deficient or inefficient glucose utilization. In one aspect, the instructions comprise instructions for treating a subject having or at risk of having ischemic/reperfusion injury, post myocardial infarction, angina, heart failure, a cardiomyopathy, peripheral vascular disease, diabetes, or lactic acidosis. In another aspect, the instructions comprise instructions for treating a subject having or at risk of having heart surgery (e.g., open heart surgery, bypass surgery, heart transplant and angioplasty).

The instructions may be on “printed matter,” e.g., on paper or cardboard within the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Kits can additionally include a buffering agent, a preservative, or a stabilizing agent. Each component of the kit can be enclosed within an individual container and all of the various containers can be within a single package.

To assist in understanding, the present invention will now be further illustrated by the following Examples. These Examples as they relate to the present invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

### Methods Used in the Studies Described in Examples A to C

The studies described in Examples A to C describe three different clinical studies of the effect of DCA when administered to patients during and/or following cardiac surgery.

The study described in Example A involved adult patients in which the effects of DCA on cardiac metabolism were studied. DCA was administered to patients undergoing elective cardiac bypass grafting surgery (CABG). This study was performed in the presence of clinically recommended dosages of hemodynamic drugs in coronary artery bypass grafts.

The study described in Example B involved the administration of a single bolus dose of DCA to pediatric patients undergoing cardiac surgery to correct congenital heart lesions. This protocol, performed in the presence of clinically recommended hemodynamic drugs, determined that the dose and amount of these agents could be decreased with DCA use.

The study described in Example C involved the use of a bolus and infusion protocol to administer DCA over a 24 hour period to pediatric patients undergoing cardiac surgery to correct congenital heart lesions. This protocol was also performed in the presence of clinically recommended hemodynamic drugs, and determined that the dose and amount of these agents could be decreased with DCA use.

### Example A

Description of Study Protocol

DCA or saline was administered to 18 patients undergoing elective cardiac bypass grafting surgery (CABG) in a double blinded randomized manner DCA (50 mg/kg in 100 ml of saline) or placebo was injected into the aortic root, immediately prior to removing aortic cross clamp. Based on the pharmacokinetics of DCA, we anticipated that this would produce a plasma concentration of approximately 1 mM. The study consisted of 8 DCA-treated patients and 10 placebo-treated patients.

1. Intervention

a. “Usual” Therapy

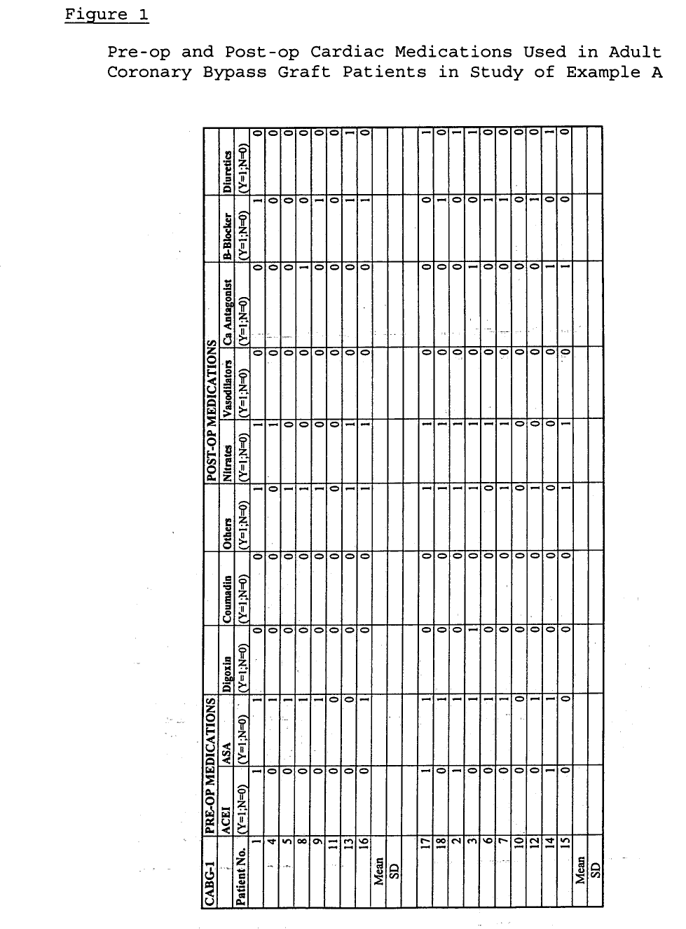

All procedures and drugs normally given for CABG patients were given routinely. A list of medications provided for these patients shown in FIG. 1.

b. “Intervention” Therapy

The intervention involved DCA (50 mg/kg) or placebo injected into the aortic root immediately prior to removing aortic cross clamp. The coded solution was made such that a dose of 1 ml/kg provides the appropriate dose of DCA or placebo. Based on the pharmacokinetics of DCA, this was expected to result in a plasma level of DCA in the therapeutic range of (1 mM). All blood samples were analyzed by HPLC for DCA concentration.

2. Sample Processing

Plasma samples were processed for DCA levels using a high performance liquid chromatography (HPLC) technique that separated the DCA from other plasma constituents. In brief, 20 μl of plasma sample was injected into a Beckman Gold HPLC containing a IonoBpher 5A column (250×4.6 mm LxID) and a AX Guard Column. The mobile phase of the column consisted of 10-3 M pyromellitate buffer (pH=4.0). The flow rate of the HPLC was set at 3.0 ml/min and the DCA eluted from the column was detected by comparing DCA elution times to acetate, monochloroacetate, and trichloroacetate standards. Heart ventricular biopsy samples were taken at 0, and 20 minutes, and at 1 hour, following release of the cross clamp and reperfusion of the heart muscle, and immediately frozen in liquid N₂. Blood samples were also taken at various intervals during the reperfusion period between 0 to 24 hours post-surgery.

PDH activity was measured in ventricular biopsies using a radioisotope procedure which determines the production of 14C-citrate formed from 14C-oxaloacetate and acetyl CoA derived from PDH (8). Blood levels of lactate, fatty acids and glucose were measured using standard enzymatic assays.

3. Statistical Analysis

Comparisons of demographics between groups were done using unpaired t-tests (continuous variables) and Chi-square tests (discrete variables). Comparison of cardiac index between groups was done using a nonparametric unpaired test. Statistical significance is defined as p<0.05. Data handling and statistical analysis was performed by the Epicore Center at the University of Alberta.

Results of Study

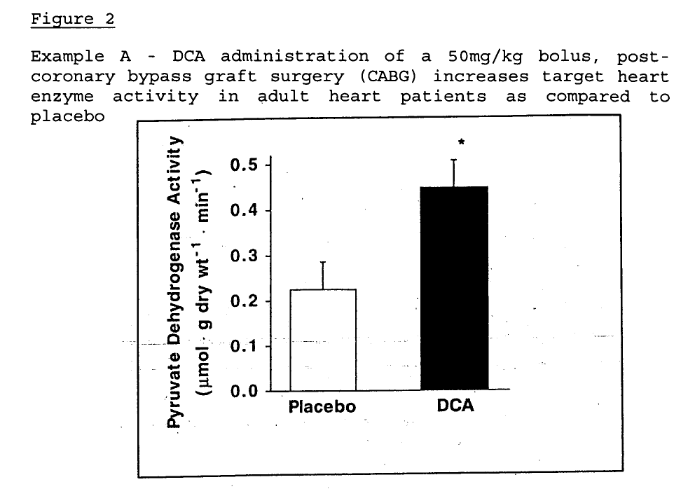

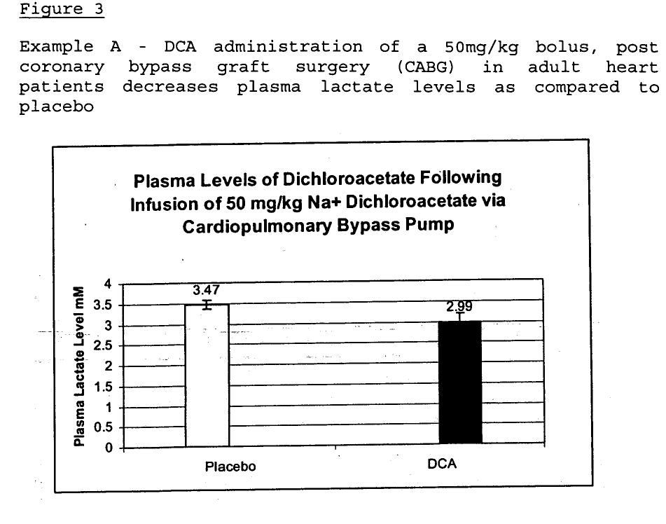

In this study in 18 adult cardiovascular surgery patients, DCA was administered as a bolus dose of 50 mg/kg to 8 adult patients in the presence of other clinically recommended doses of hemodynamic drugs (FIG. 1). DCA was administered immediately prior to restoration of coronary blood flow following the cardiac procedure. In patients treated with DCA, compared to placebo, there was a significant increase in PDH activity in heart muscle biopsies taken in the early reperfusion period (FIG. 2). DCA also significantly decreased lactate levels (FIG. 3), indicating that DCA increases carbohydrate oxidation during reperfusion. There was a single mortality in the placebo group and no mortalities the DCA group.

Plasma levels of DCA were also measured in patients at 1 hour following administration of DCA. Plasma levels of DCA were approximately 1 mM, a concentration we have shown to be efficacious in stimulating glucose oxidation in experimental animal studies (9, 16).

TABLE III
Plasma Levels of Dichloroacetate Following Infusion of 50 mg/kg
Na⁺ Dichloroacetate via Cardiopulmonary Bypass Pump
Plasma Dichloroacetate Levels
(mM)
(n = 8)
0.948 ± 0.061

Combined, the data in this study of adult patients demonstrated that our dosing protocol: 1) resulted in a therapeutic level of DCA in the critical early period of reperfusion post cardiac surgery, and 2) this dose of DCA increased cardiac PDH activity and lowers circulating plasma lactate levels.

### Example B

Description of Study Protocol

This study was a randomized, placebo-controlled, double blinded, single surgeon, study of the use of DCA in 40 high-risk pediatric patients requiring heart surgery to connect complex congenital heart lesions.

1. Study Population

In this trial, 40 children were recruited to participate in a single surgeon study, of which 18 received DCA and 22 received placebo. The 1995 power calculations were based on separation of the CPB-1 trial of n=40 patients.

2. Inclusion Criteria

a. Age less than 1 year.
b. Consent from parent or guardian.
C. Requirement for open-heart surgery to correct complex congenital heart lesions (e.g., such as Tetralogy of Fallot).
d. Agreement of the surgeon, anesthetist and cardiologist.
e. Significant non cardiac complications precluding study protocol implementation.
   3. Exclusion Criteria
a. Lack of parental consent.
b. Refusal for entry from surgeon or anesthetist or cardiologist
   4. Randomization, Data Collection, and Blinding Procedures

Computerized randomization of study medications were performed by the Epicore Centre at the University of Alberta. The patients and all study personnel were blinded throughout the study. Unblinding was set into the procedures only if, in the opinion of the patient's physician or study personnel, information concerning the identity of the study drug was essential for the patients' safety reasons.

5. Intervention

a. “Usual” Therapy

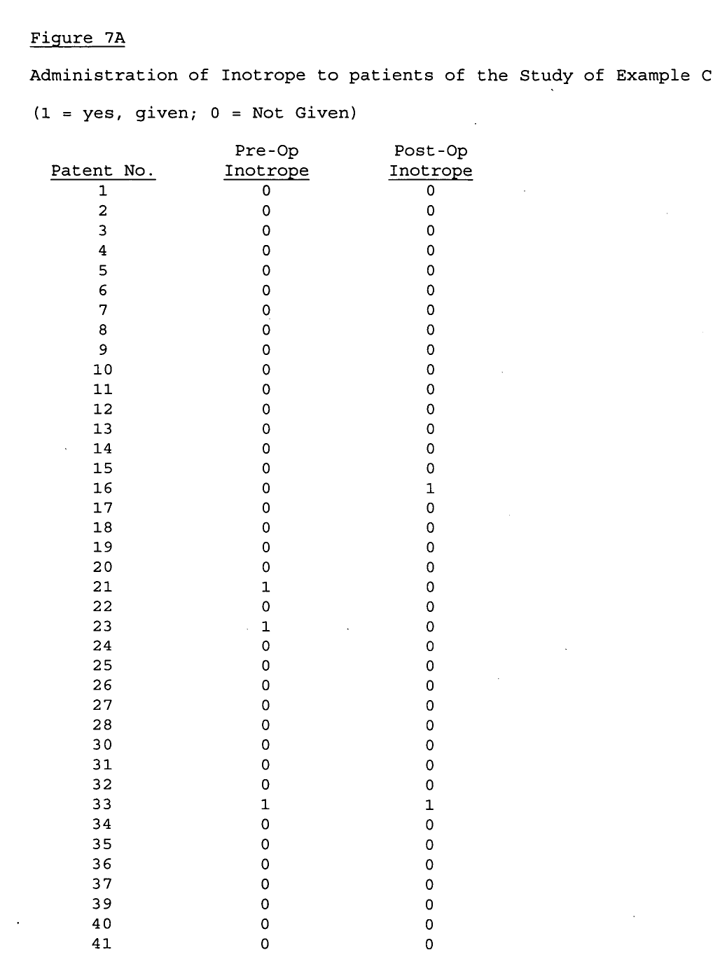

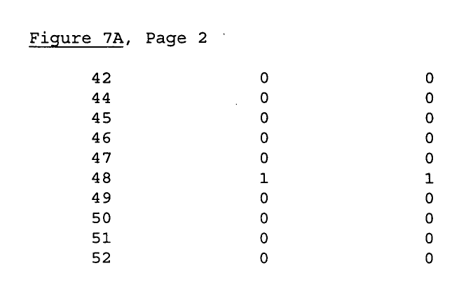

All procedures and drugs normally given for infants undergoing cardiopulmonary bypass were given routinely. A list of medications provided is shown in FIG. 7B.

b. “Intervention” Therapy

The intervention involved DCA (50 mg/kg) or placebo injected into the aortic root immediately prior to removing aortic cross clamp. The coded solution was made such that a dose of 1 ml/kg provides the appropriate dose of DCA or placebo. Based on the pharmacokinetics of DCA, this was expected to result in plasma levels of DCA in the therapeutic range of (1 mM). All blood samples were analyzed by HPLC for DCA concentration.

6. Sample Collections

Arterial blood samples were obtained from patients at the following times:
a. Immediately after the insertion of arterial line in operating room, i.e., the beginning of surgery.
b. Thirty minutes after the bolus of DCA was given, whether or not cardiopulmonary bypass had been discontinued.
c. One hour after discontinuing cardiopulmonary bypass.
d. Six hours after discontinuing cardiopulmonary bypass.
e. Twelve hours after discontinuing cardiopulmonary bypass.
f. Twenty four hours after discontinuing cardiopulmonary bypass.
   7. Sample Processing

Blood samples were collected from indwelling arterial lines into citrate-containing tubes (0.5 ml blood samples). The samples were spun in the microfuge, the plasma separated, and frozen immediately for later analysis. All plasma samples were stored at −80 degrees centigrade, until further processing. Plasma glucose and lactate were determined using a Sigma glucose kit and a spectrophotometric assay involving lactate dehydrogenase respectively. Plasma fatty acid levels were measured using an ELISA system and WAKO free fatty acid kit.

8. Inotrope Drug Score

In both the operating room at the end of cardiopulmonary bypass and in the intensive care unit, parenteral drugs were scored on an hourly basis with 1 point allotted for each level for each bolus or infusion given within the previous hour for the first 24 hours post-operatively. Thus, at the end of 24 hours high scores indicated poorer cardiac function.

9. Validation of Index

In this study, we anticipated a 30% decrease in Inotrope Score at the 1 hour interval.

10. Ascertainment of Response Variables

a. Data Collection

The drug score chart in the operating room was filled out by the anesthetist. In the pediatric intensive care unit, the research coordinator was responsible for completing drug score charts, corroborated by nursing staff, ICU staff and physicians. Fatty acids, glucose, DCA, and lactate levels were determined with technicians blinded as to treatment category.

b. Data Monitoring and Safety issues

Careful attention was paid to safety precautions in this study. A data monitoring committee had the authority to terminate the study should have serious adverse side effects occurred. In previous pilot studies, no adverse effects of DCA were noted.

c. Data Analysis

DCA was deemed beneficial if Inotrope Score was significantly lower in the intervention patients than in placebo patients.

11. Statistical Analysis

Comparison of demographics between groups was done using unpaired t-tests (continuous variables) and Chi-square tests (discrete variables). Comparison of Cardiac functional Index between groups was done using a nonparametric unpaired test. Statistical significance is defined as p<0.05. Data handling and statistical analysis was performed by the Epicore Center.

Results of Study

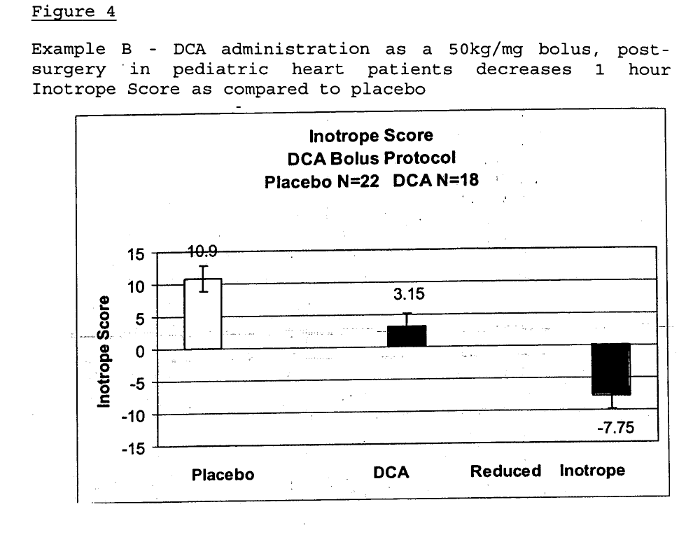

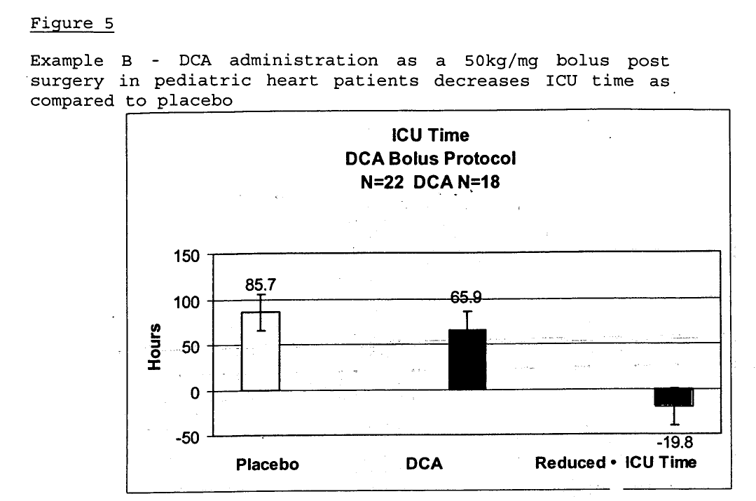

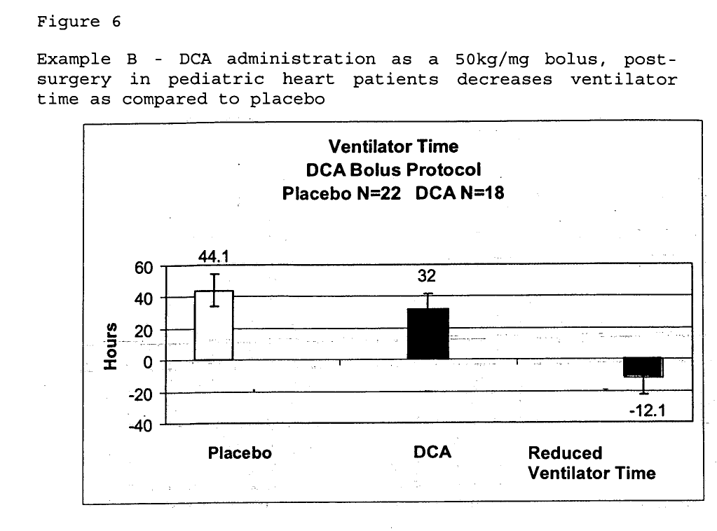

DCA administration significantly reduced the need for inotropic drugs during the critical first 1 hour period following surgery (FIG. 4). Data from this bolus administration of DCA to pediatric patients (40) also demonstrates that post surgical DCA administration reduces ICU time (FIG. 5) and ventilator time (FIG. 6). In this protocol which had 40 pediatric patients, 18 pediatric patients received a DCA bolus of 50 mg/kg. Echocardiography in the DCA patients (35% versus 26%) demonstrated better shortening fraction as compared to placebo patients.

### Example C

Description of Study Protocol

This study was a randomized, placebo-controlled, double blinded, single surgeon, study of the use of DCA in 51 high-risk pediatric patients requiring heart surgery to correct complex congenital heart lesions.

1. Study Population

In this trial, 53 infants were recruited to participate in a study, of which 51 patients met inclusion criteria after parental consent. Two dosing groups resulted from the study team changing the dosing protocol after the data from the initial 10 patients were analyzed. The data from patients number 1 to number 10 was analyzed for DCA therapeutic blood levels and therapeutic effect. A recommendation by the research team was made to increase the bolus dose of DCA and to decrease the infusion dose of DCA to maintain a DCA dose range of 1 mM for 24 hours. The intent was to administer the new protocol at patient number 20. The 1997 Epicore power calculations were based on separation of the study groups patients into group A of n=20 patients and Group B of n=31 patients

Half of each group received different dosages of DCA and the other half received placebo in a double blinded, randomized fashion. Candidates for entry into the study were recruited from weekly surgical lists and from notification by the cardiac surgeon.

2. Inclusion Criteria

a) Age less than 1 year.
b) Consent from parent or guardian.
c) Requirement for open-heart surgery to correct complex congenital heart lesions (e.g., tetralogy of Fallot).
d) Agreement of the surgeon, anesthetist, and cardiologist.
   3. Exclusion Criteria
a) Lack of parental consent.
b) Refusal for entry from surgeon or anesthetist or cardiologist.
c) Significant non cardiac complications precluding study protocol implementation.
   4. Randomization, Data Collection, and Blinding Procedures

Computerized randomization of study medications were performed by the Epicore Centre. The patients and all study personnel were blinded throughout the study. Unblinding was set into the procedures only if, in the opinion of the patient's physician or study personnel, information concerning the identity of the study drug was essential for the patients' safety reasons.

5. Intervention

a) “Usual” Therapy

All procedures and drugs normally given for infants undergoing cardiopulmonary bypass were given routinely. A list of medications provided in shown in FIGS. 7A and 7B.

b) “Intervention” Therapy

The interventions in the two groups of this study were as follows:

(i) Group A

DCA (50 mg/kg) or placebo was injected into the aortic root immediately prior to removing aortic cross clamp. The coded solutions were made such that a dose of 1 ml/kg provided either a DCA therapeutic level of 1 mM plasma concentration of DCA, or a placebo solution. Immediately thereafter, an infusion of DCA at 25 mg/kg/hr or placebo in the same volume was initiated and run for 24 hours. Based on the pharmacokinetics of DCA, this was expected to maintain plasma levels of DCA in the therapeutic range of (0.2-1 mM). However, the plasma concentrations of DCA were below 1 mM after the first hour interval, and that the 24 hour plasma concentrations were elevated above 1 mM DCA levels at the 24 hour interval, a decision was made to modify the dosing protocol. This change in dosing protocol was approved by the Ethics Committee, but not implemented until patient number 24. All blood samples were analyzed by HPLC for DCA concentration.

(ii) Group B

DCA (100 mg/kg) or placebo was injected into the aortic root immediately prior to removing aortic cross clamp. The coded solutions were made such that a dose of 1 ml/kg provided either a DCA therapeutic level of 1 mM plasma concentration of DCA, or a placebo solution. Immediately thereafter, an infusion of DCA at 12.5 mg/kg/hr or placebo in the same volume was initiated and run for 24 hours. Based on the pharmacokinetics of DCA, this was expected to maintain plasma levels of DCA in the therapeutic range of (0.2-1 mM). All blood samples were analyzed by HPLC for DCA concentration.

6. Sample Collections

Arterial blood samples were obtained from patients at the following times:
a. Immediately after the insertion of arterial line in operating room (i.e., at beginning of surgery).
b. Thirty minutes after the bolus of DCA has been given, whether or not cardiopulmonary bypass has been discontinued.
c. One hour after discontinuing cardiopulmonary bypass.
d. Six hours after discontinuing cardiopulmonary bypass
e. Twelve hours after discontinuing cardiopulmonary bypass.
f. Twenty four hours after discontinuing cardiopulmonary bypass.
   At the end of 24 hours, the DCA or placebo infusion was discontinued.
   7. Sample Processing

Blood samples were collected from indwelling arterial lines into citrate-containing tubes (0.5 ml blood samples). The samples were spun in the microfuge, the plasma separated, and frozen immediately for later analysis. All plasma samples were stored at −80 degrees centigrade, until further processing. Plasma glucose and lactate were determined using a Sigma glucose kit and a spectrophotometric assay involving lactate dehydrogenase respectively. Plasma fatty acid levels were measured using an ELISA system and WAKO free fatty acid kit.

8. Inotrope Drug Score

In both the operating room at the end of cardiopulmonary bypass and in the intensive care unit, parenteral drugs were scored on an hourly basis with 1 point allotted for each level for each bolus or infusion given within the previous hour within the first 24 hours post-operatively. Thus at the end of 24 hours, high scores indicated poorer cardiac function.

9. Validation of Index

In this study, we anticipated a 30% decrease in Inotrope Score. Our intent was to maintain good or improve contractile function through the 24 hour period as compared to placebo, by providing an infusion of DCA throughout the 24 hour period following a bolus administration of DCA. By improving cardiac function, we anticipated a reduction in ICU time per patient.

10. Ascertainment of Response Variables

a) Data Collection

The drug score charts in the operating room were filled out by the anesthetist. In the pediatric intensive care unit, the research coordinator was responsible for completing drug score charts, corroborated by nursing, ICU flow sheets, and doctor's orders. Fatty acids, glucose, DCA, and lactate levels were determined with technicians blinded as to treatment category.

b) Data Monitoring and Safety Issues

Careful attention was paid to safety precautions in this study. A data monitoring committee has the authority to terminate the study should have serious adverse side effects occurred. In previous studies, no adverse effects of DCA were noted.

c) Data Analysis

DCA was deemed beneficial if Inotrope Score was significantly lower in the Intervention patient compared to the placebo patients.

11. Statistical Analysis

Comparison of demographics between groups was done using unpaired t-tests (continuous variables) and Chi-square tests (discrete variables). Comparison of Cardiac functional Index between groups was done using a nonparametric unpaired test. Statistical significance is defined as p<0.05. Data Handling and statistical analysis was performed by the Epicore Center.

Results of Study

Since DCA has a short-half life in the body this study was initiated in pediatric patients where a DCA bolus and infusion protocol was used over a 24 hour period in the presence of other clinically recommended doses of hemodynamic drugs (FIGS. 7A and 7B). The goal of this study was to maintain therapeutic levels of DCA over a 24 hour period because it is known that poor myocardial contractility and high lactate levels (10) persist for up to 24 hours in children after open heart surgery.

In a double-blinded randomized clinical trial involving 51 pediatric patients (age 3 days to 12 years) requiring open-heart surgery were given either a DCA bolus or placebo followed by an infusion of DCA for 24 hours. During the course of this study, after the protocol was administered to first 10 patients in the Group A (out of the 24 patients), it became clear that the original Group A infusion rate produced concentrations of DCA in excess of 1 mM by 24 hours. We therefore modified the Group A bolus infusion protocol, as described in the “Methods” section for Group B in this document. In the Group A, 12 patients received a DCA bolus of 50 mg/kg followed by an infusion of DCA (25 mg/kg/hr) for 24 hours. In the Group B, 14 patients received a DCA bolus of 100 mg/kg followed by an infusion of DCA (12.5 mg/kg/hr) for 24 hours.

The following observations were as follows from this study: There was also a trend toward lower Inotrope Scores in the DCA groups over the 24 hour period as compared to placebo. There was also a trend toward less Intensive Care Unit (ICU) days in the DCA groups over the 24 hour period as compared to placebo. There was also a trend toward less ventilator time in DCA Group A as compared to placebo. This decrease in ventilator time was lower than what was observed from both the DCA Group B protocol and the DCA protocol of the Study described in Example B. Greater differences in ICU Time were observed in patients who had poorer initial function with more complex conditions and surgical procedures, which suggests that DCA may be more beneficial then placebo for these patients.

A subsequent review of the data obtained from the 51 patients in this study revealed the following. The change in dosing protocol in this study was initiated at patient number 25 (and not at patient number 20 as anticipated at the time of request for protocol change), and that the actual number of patients allocated to each group was 24 patients for the Group A, and 27 patients for the Group B. Subsequent review of the patient records and data of the 24 older, pediatric patients in the Group A revealed the inclusion of 1 infusion pump failure case. In the 27 younger, pediatric patients in the Group B, there were 3 infusion pump failure cases. In total, 4 only infusion pump failure cases were excluded in the subsequent data compilation. As a result of the infusion pump failure modifications, the final allocation of patients included in the final two groups in this document were as follows: Group A of n=23 patients and Group B of n=24 patients. The drug scoring compilation of data was set up for Inotrope Scoring, and not sodium bicarbonate scores. (Sodium bicarbonate is not considered an inotropic drug.) As a result, sodium bicarbonate scores were removed in the final compilation of the Inotrope Scoring data. Clinically recommended doses of other hemodynamic drugs administered to both the placebo and drug patient groups were noted (see FIGS. 7A and 7B).

1. Inotrope Score

In this study, a trend in decreased Inotrope Score over the 24 hour period post-surgery was shown, similar to what was observed in the study described in Example B (FIG. 4) over the 1 to 4 hour period. A trend to a decreased inotrope use was noted in both Group A, and Group B patients receiving DCA. Data from the study, Group A (FIG. 8) and Group B (FIG. 9), show the effects of DCA bolus/infusion administration using the two dosing protocols on Inotrope Score over the 24 hour period. In the Group B (FIG. 9), the decrease in Inotrope Score (an average decrease of 51% per patient as compared to placebo) was greater than the results from the Group A (an average decrease of 44% per patient as compared to placebo). It should be noted that in this study, all patients in the A and B Groups on average received lower Inotrope Scores than those reported for the patients in the study described in Example B patients, due to the involvement of a different cardiac surgeon.

2. ICU Time

In this study Group A, and Group B, a trend in decreased ICU time was similar to what was observed in the study described in Example B (FIG. 5). A trend to decreased ICU time over the 24 hour period was noted in both Group A and Group B patients receiving DCA. Data from the Group A (FIG. 10) and Group B (FIG. 11), show the effects of DCA bolus/infusion administration on ICU time, using the two different dosing protocols. In Group A, the reduction in ICU time (a decrease of 60 hours or 41% as compared to placebo), was greater than the results from both the study described in Example B (a decrease of 19 hours or 23% compared to placebo), and the Group B (a decrease of 50 hours, or 40% compared to placebo).

3. Ventilator Time

Data from the Group A and Group B showed that the trend in decreased ventilator time was similar to what was observed in the study described in Example B (FIG. 6). A trend to decreased ventilator time over the 24 hour period was noted in both Group A and Group B patients receiving DCA. Data from the Group A (FIG. 12) and Group B (FIG. 13) show the effects of DCA bolus/infusion administration on ventilator time using the two different dosing protocols. In Group A, the reduction in ventilator time (a decrease of 46 hours or 47% as compared to placebo) was greater than the results from both the study described in Example B (a decrease of 12 hours or 27% compared to placebo) and the Group B (a decrease of 18 hours or 23% compared to placebo).

### CONCLUSION

In summary, our findings support our first outcome measure to improve cardiac function through a surrogate measurement score for “cardiac index” which showed a reduced need for inotropes, and a reduced ICU time and reduced ventilator time post-surgery as compared to placebo. We have established through our three studies that DCA improves cardiac function and provides cardioprotection during reperfusion in both neonates and adults as additive and/or in combination therapy with hemodynamic drugs. The data from these studies supports the use of DCA as a therapeutic approach for treating both the adult and pediatric cardiac surgical patients. The data also supports the combined used of DCA with inotropes in the presence of other clinically recommended doses of hemodynamic drugs, and demonstrates that DCA can lessen the amount of inotropes needed post-surgery.

### REFERENCES

1. Bersin R M, Wolfe C, Kwasman M, Lau D, Klinski C, Tanaka K, Khorrami P. Henderson G N, DE Marco T, Chatterjee K: Improved hemodynamic function and mechanical efficiency in congestive heart failure with sodium dichloroacetate. JACC 1994;23(7): 1617-1624.
2. Chandler B M, Sonnenblick E H, Pool F E: Mechanochemistry of cardiac muscle III. Effects of norepinephrine on the utilization of high energy phosphates. Circ Res 1968; 22: 729-735.
3. Suga H, Hisano R, Goto Y, Yamada 0, Igarashi Y: Effect of positive inotropic agents on the relation between oxygen consumption and systolic pressure volume area in canine left ventricle. Circ Res 1983; 53:306-318.
4. Hasenfuss G, Mulieri LA, Allen PD, Just H, Alpert N R: Influence of isoproterenol and ouabain on excitation-contraction coupling, crossbridge function and energetics in failing human myocardium. Circulation 1996; 94:3 155-3160.
5. Lopaschuk G D: Alterations in fatty acid oxidation during reperfusion of the heart after myocardial ischemia. Am J Cardiol 1997; 80 (3A): 1A-16A.
6. Stanley W C, Lopaschuk G D, Hall J L, McCormack J G: Regulation of myocardial carbohydrate metabolism under normal and ischemic conditions potential for pharmacological interventions Cardiovascular Res 1997, 33 243-257
7. Lopaschuk G D, Saddik M: The relative contribution of glucose and fatty acids to ATP production in hearts reperfused following ischemia. Mol Cell Biochem 1992; 116:1 11-116.
8. Collins-Nakai R L, Noseworthy D, Lopaschuk G D: Epinephrine increases ATP production in hearts by preferentially increasing glucose metabolism. Am J Physiol 1994; 267:H1862-H1871.
9. Lopaschuk G D, Wambolt R B, Barr R L: An imbalance between glycolysis and glucose oxidation is a possible explanation for the detrimental effects of high levels of fatty acids during aerobic reperfusion of ischemic hearts. J Pharmacol Exp Ther 1993; 264: 135-144.
10. Lopaschuk G D, Collins-Nakai R, Olley P M, Montague T J, McNeil G, Gayle M, Penkoske P, Finegan B A: Plasma fatty acid levels in infants and adults after myocardial ischemia. Am Heart J 1994; 128:61-67
11. Vik-Mo H, Mjos O D: Influence of free fatty acids on myocardial oxygen consumption and ischemic injury. Am J Cardiol 198 1;48:361-364.
12. Lopaschuk G D: Treating ischemic heart disease by pharmacologically improving cardiac energy metabolism. Am J Cardiol 1998; 82: 14K-17K.
13. Itoi T, Lopaschuk G D: The contribution of glycolysis, glucose oxidation, lactate oxidation, and fatty acid oxidation to ATP production in isolated biventricular working hearts from 2-week old rabbits. Ped Res 1993; 34(6) :735-741.
14. Stacpoole P W, Henderson G N, Yan Z, Cornett R, James Mo.: Pharmacokinetics, metabolism and toxicology of dichloroacetate. Drug Met Rev 1998; 30(3): 499-538. Saddik M, Gamble J, Witters L A, Lopaschuk G D: Acetyl-CoA carboxylase regulation of fatty acid oxidation in the heart. J Biol Chem 1993; 268(34):25836-25845.
16. Lui B, Clanachan A S, Schulz R, Lopaschuk G D: Cardiac efficiency is improved after ischemia by altering both the source and fate of protons. Circ Res 1996; 79:940-948.
17. McVeigh J J, Lopaschuk G D: Dichloroacetate stimulation of glucose oxidation improves recovery of ischemic rat hearts Am J Physiol 1990, 259H1079-H1085
18. Bersin R M, Stacpoole P W: Dichloroacetate as metabolic therapy for myocardial ischemia and failure. Am Heart J 1997; 134:841-855.
19. Lopaschuk G D, Collins-Nakai RL, Itoi T: Developmental changes in energy substrate use by the heart. Cardiovasc Res 1992; 26: 1172-1180.
20. Makinde A, Kantor P F, Lopaschuk G D: Maturation of fatty acid and carbohydrate metabolism in the newborn heart. Mol Cell Biochem 1998; 188:49-56.
21. Itoi T, Huang L, Lopaschuk G D: Glucose use in neonatal rabbit hearts reperfused after global ischemia. Am J Physiol 1993; 265:H427-H433.
22. Lopaschuk G D, Spafford Mass.: Energy substrate utilization by isolated working hearts from newborn rabbits. Am J Physiol 1990; 258:H1274-H1280.
23. Saiki Y, Lopaschuk G D, Dodge K, Yamaya K, Morgan C, Rebeyka T M: Pyruvate augments mechanical function via activation of the pyruvate dehydrogenase complex in reperfused ischemic immature rabbit hearts. J Surg Res 1998; 79: 164-169.

1. A method of maintaining or improving cardiac function during or following a cardiac function disturbing event or a cardiac metabolism disturbing event in a patient which comprises administering to said patient a cardioprotective amount of dichloroacetate (DCA) and an inotropic drug.

2. A method according to claim 1 wherein said cardiac function disturbing event or cardiac metabolism disturbing event is an ischemic event.

3. A method according to claim 2 wherein said cardiac function disturbing event or cardiac metabolism disturbing event is an acute myocardial infarction (AMI).

4. A method according to claim 1 wherein said cardiac function disturbing event or cardiac metabolism disturbing event is acute heart failure.

5. A method according to claim 1 wherein said cardiac function disturbing event or cardiac metabolism disturbing event is caused by hemorrhagic shock, hypoxia or trauma.

6. A method according to claim 1 wherein said cardiac function disturbing event or cardiac metabolism disturbing event is due to cardiomyopathy.

7. A method according to claim 6 wherein said cardiomyopathy is diabetic myopathy.

8. A method according to claim 1 wherein said cardiac function disturbing event or cardiac metabolism disturbing event is due to HIV infection.

9. A method according to claim 1 wherein said cardiac function disturbing event or cardiac metabolism disturbing event is due to malaria.

10. A method according to claim 1 wherein said cardiac function disturbing event or cardiac metabolism disturbing event is due to an acute coronary syndrome (ACS).

11. A method according to claim 10 wherein said ACS is post-AMI, post Percutaneous Transluminal Coronary Angioplasty (PTCA) or angina.

12. A method according to claim 1 wherein said cardiac function disturbing event or cardiac metabolism disturbing event is shock.

13. A method according to claim 12 wherein shock is secondary to hemorrhage, hypoxia, trauma or sepsis.

14. A method according to claim 1 wherein said cardiac function disturbing event or cardiac metabolism disturbing event is associated with diabetes.

15. A method of maintaining cardiac function at a predetermined level in a patient during or following a cardiac function disturbing event or a cardiac metabolism disturbing event and decreasing said patient's need for inotropic drugs which comprises administering to said patient a cardioprotective amount of DCA.

16. A method according to claim 15 wherein DCA and inotropic drug are administered in combination.

17. A method according to claim 15 wherein DCA is administered within about 15 minutes of administering an inotropic drug.

18. A method according to any of claims 1, 15, 16 or 17 wherein said inotropic drug is selected from the group consisting of dobutamine, epinephrine, dopamine, norepinephrine, phentolamine, digoxin, amrinone, milrnone, and enoximone.

19. A method according to any of claims 1, 15, 16 or 17 wherein DCA is administered to said patient in a bolus of at least about 100 mg/kg followed by continuous infusion of DCA of at least about 25 mg/kg/hour for at least about 10 hours.

20. A method according to claim 19 wherein said infusion of DCA is for at least about 24 hours.

21. A method according to claim 1 wherein said inotropic drug is selected from the group consisting of a beta-adrenergic receptor agonist, a photodiesterase 3 (“PDE3”) inhibitor, an agent which increases cyclic AMP levels, a sodium hydrogen (Na⁺, H⁺) exchange inhibitor, and a sodium calcium (Na⁺/Ca²⁺) exchange blocker.

22. A method according to claim 21 wherein said inotropic drug is an Na⁺/Ca²⁺ exchange blocker.

23. A method according to claim 1 wherein said inotropic drug is a non-adrenegic vasopressor.

24. A method according to claim 23 wherein said inotropic drug is vasopressin.

25. A method according to claim 1 wherein said inotropic drug is an alpha-2-adrenegic agonist.

26. A method according to claim 25 wherein said inotropic drug is moxonidine or clonidine.

27. A method according to claim 1 wherein said inotropic drug is an endothelin 1 (ET-1) antagonist.

28. A method according to claim 27 wherein said ET-1 antagonist is bosetan or tezosentan.

29. A method according to claim 1 wherein said inotropic drug is an ion channel blocker.

30. A method according to claim 29 wherein said ion channel blocker is an Na⁺ pump inhibitor or an Na⁺, H+ exchange inhibitor.

31. A method according to claim 1 wherein said inotropic drug is a calcium-sensitizing agent.

32. A method according to claim 31 wherein said inotropic drug is levosimendan.

33. A method according to claim 1 wherein said inotropic drug is a calcium channel blocker.

34. A method according to claim 33 wherein said inotropic drug is diltiazem or nifedipine.

35. A method according to claim 1 wherein said inotropic drug is an angiotensin converting enzyme (“ACE”) inhibitor.

36. A method according to claim 35 wherein said inotropic drug is quinaprilat.

37. A method according to claim 1 wherein said inotropic drug is a PDE3 inhibitor.

38. A method according to claim 37 further comprising administering a beta-adrenegic receptor agonist with said inotropic drug.

39. A method according to claim 1 wherein said inotropic drug is an agent which increases cyclic AMP levels.

40. A method according to claim 1 wherein said inotropic drug is an Na⁺, K⁺-ATPase inhibitor or a cardiac glycoside.

41. A method according to claim 40 wherein said inotropic drug is vandate, 2-methoxy-3,8,9-dihydroxy coumestan or digoxin.

42. A method according to claim 1 further comprising administering an agent which increases arginine levels in combination with DCA and said inotropic drug.

43. A method according to claim 1 wherein said inotropic drug is administered in an amount effective to maintain or improve cardiac function.

44. A method of treating an ischemic, hypoxic or metabolic event or an event resulting in cardiac dysfunction in a patient which comprises administering to said patient a cardioprotective amount of dichloroacetate (“DCA”) and an inotropic drug.

45. A method according to claim 44 wherein said event is due to a cardiac surgical procedure, percutaneous intervention, acute myocardial infarction or an acute coronary syndrome.

46. A method according to claim 45 wherein said event is a cardiac surgical procedure.

47. A method according to claim 45 wherein said event is an acute coronary syndrome selected from cardiogenic shock, hemorrhagic shock and trauma.

48. A method according to claim 44 wherein said event results from sepsis, HIV or malaria.

49. A method according to claim 44 wherein said event occurs following cancer chemotherapy.

50. A method according to claim 44 wherein said event is due to or results from angina, hypertension, pulmonary hypertension, diabetic cardiomyopathy, cardiomyopathy, congestive heart failure or diabetes.

51. A method according to claim 44 wherein said event results in cognitive impairment.

52. A method according to claim 44 wherein said cardioprotective amount of DCA comprises a bolus of at least about 50 mg/kg followed by infusion of at least about 12.5 mg/kg/hour.

53. A method according to claim 44 wherein said cardioprotective amount of DCA comprises a bolus of at least about 100 mg/kg followed by infusion of at least about 25 mg/kg/hour.

54. A method according to claim 53 wherein DCA is infused for at least about 10 hours.

55. A method according to claim 53 wherein DCA is infused for at least about 24 hours.

56. A pharmaceutical composition comprising a cardioprotective amount of DCA and an inotropic drug selected from the group consisting of a beta-adrenergic receptor agonist, a PDE3 inhibitor, an agent which increases cAMP levels; a Na⁺, H+ exchange inhibitor; a Na⁺, Ca²⁺ exchange blocker; a non-adrenergic vasopressor; an alpha-2-adrenergic agonist; an ET-1 antagonist; an ion channel blocker; a calcium-sensitizing agent; a calcium channel blocker; an ACE inhibitor; a Na⁺, K⁺-ATPase inhibitor; a Na⁺, K⁺ exchange inhibitor; a cardiac glycoside; and a sympathomimetic, and a pharmaceutically acceptable carrier.

57. A pharmaceutical composition according to claim 56 wherein said inotropic drug is selected from the group consisting of a beta-adrenergic receptor agonist, a PDE3 inhibitor, an agent which increases cAMP levels, a Na⁺, H⁺ exchange inhibitor; and a Na⁺/Ca²⁺ exchange blocker.

58. A pharmaceutical composition according to claim 57 wherein said inotropic drug is an Na⁺/Ca²⁺ exchange blocker.

59. A pharmaceutical composition according to claim 56 wherein said inotropic drug is a non-adrenegic vasopressor.

60. A pharmaceutical composition according to claim 59 wherein said inotropic drug is vasopressin.

61. A pharmaceutical composition according to claim 56 wherein said inotropic drug is an alpha-2-adrenegic agonist.

62. A pharmaceutical composition according to claim 61 wherein said inotropic drug is moxonidine or clonidine.

63. A pharmaceutical composition according to claim 56 wherein said inotropic drug is an endothelin 1 (ET-1) antagonist.

64. A pharmaceutical composition according to claim 63 wherein said ET-1 antagonist is bosetan or tezosentan.

65. A pharmaceutical composition according to claim 56 wherein said inotropic drug is an ion channel blocker.

66. A pharmaceutical composition according to claim 65 wherein said ion channel blocker is an Na⁺ pump inhibitor or an Na⁺,H⁺ exchange inhibitor.

67. A pharmaceutical composition according to claim 56 wherein said inotropic drug is a calcium-sensitizing agent.

68. A pharmaceutical composition according to claim 67 wherein said inotropic drug is levosimendan.

69. A pharmaceutical composition according to claim 56 wherein said inotropic drug is a calcium channel blocker.

70. A pharmaceutical composition according to claim 69 wherein said inotropic drug is diltiazem or nifedipine.

71. A pharmaceutical composition according to claim 56 wherein said inotropic drug is an angiotensin converting enzyme (“ACE”) inhibitor.

72. A pharmaceutical composition according to claim 71 wherein said inotropic drug is quinaprilat.

73. A pharmaceutical composition according to claim 56 wherein said inotropic drug is a PDE3 inhibitor.

74. A pharmaceutical composition according to claim 73 further comprising a beta-adrenegic receptor agonist.

75. A pharmaceutical composition according to claim 56 wherein said inotropic drug is an agent which increases cyclic AMP levels.

76. A pharmaceutical composition according to claim 56 wherein said inotropic drug is an Na⁺, K⁺-ATPase inhibitor or a cardiac glycoside.

77. A pharmaceutical composition according to claim 76 wherein said inotropic drug is vandate, 2-methoxy-3,8,9-dihydroxy coumestan or digoxin.

78. A pharmaceutical composition according to claim 56 further comprising administering an agent which increases arginine levels in combination with DCA and said inotropic drug.

79. A kit containing a pharmaceutical composition according to any of claims 56 to 78.

80. A kit according to claim 79 wherein said kit comprises a label or packaging insert containing instructions for use, in vitro, in vivo or ex vivo and components of said kit.
